(19) United States
(12) Patent Application Publication
(10) Pub. No.: US 2014/0017333 A1
(43) Pub. Date: Jan. 16, 2014

(54) METHOD FOR FERMENTING ANTLERS, VENISON, OR DEER BONES USING MUSHROOMS, AND RESULTANT FERMENTED PRODUCTS

(71) Applicants: Young-Hee Kim, Gangwon-do (KR); Jung-Sun Kim, Gangwon-do (KR)

(72) Inventors: Young-Hee Kim, Gangwon-do (KR); Jung-Sun Kim, Gangwon-do (KR)

(21) Appl. No.: 14/003,539

(22) PCT Filed: Feb. 24, 2012
(86) PCT No.: PCT/KR2012/001418

§ 371 Date: Sep. 6, 2013

(30) Foreign Application Priority Data
Apr. 5, 2011 (KR) 10-2011-0031241

(51) Int. Cl. A61K 35/32; C12P 1/02

(52) U.S. Cl. CPC A61K 35/32; C12P 1/02
USPC 424/549; 435/171

(57) ABSTRACT

The present invention relates to a method of fermenting velvet antler (or venison or deer bone) together with a mushroom concentration in order to increase the efficacy thereof, and a fermentation product obtained thereby. In accordance with the present invention, velvet antler is fermented in a state in which it was not concentrated or ground, and the shape thereof is maintained after fermentation. Thus, the loss of velvet antler is insignificant, and the fermented velvet antler has a high efficacy and can be used in various shapes in subsequent processes.

### CROSS-REFERENCE TO RELATED APPLICATION

This application is a Section 371 National Stage Application of International Application No. PCT/KR2012/001418, filed Feb. 24, 2012, the contents of which is hereby incorporated by reference in its entirety.

### BACKGROUND OF THE INVENTION

1. Technical Field

The present invention relates to a method of fermenting velvet antler (or venison or deer bone; hereinafter the same shall apply) with a mushroom concentrate, and a fermentation product produced thereby. More particularly, the present invention relates to a method of fermenting velvet antler using one or more mushroom concentrates selected from among Polyozellus multiplex, Sarcodon asparatus, and Inonotus obliquus concentrates, and a fermentation product produced thereby.

2. Background Art

As used herein, the term “velvet antler” (Latin name: Cervi parvum cornu) refers to one obtained by cutting and drying the non-ossified young horn of bucks, such as Cervus nippon Temminck, Cervus elaphus Linne or others, which belong to the family Cervidae. The non-ossified young horn is referred to as velvet antler, an ossified horn is referred to as a deer horn, and a horn that is naturally detached in the next year is referred to as a detached horn. Velvet antlers are classified into Cervus elaphus Linne, Cervus nippon Temminck, Cervus Canadensis (Siberia), red deer (New Zealand) and reindeer velvet antlers in accordance with the production district and kind of deer.

In Chinese medicine, velvet antler has been regarded as the best hematic tonic agent. As described in the literature such as Donguibogam, it is known that velvet antler has many effects, including tonic, hematic, aphrodisiac, analgesic, hematopoietic, growth promotion, heart failure treatment and function increasing effects, as well as the effects of recovering from fatigue, enhancing the vital energy of the body and enhancing the urination function of the kidneys. Further, the results of studies performed by animal tests, clinical tests and the like show that velvet antler has a wide range of therapeutic effects, including the effect of promoting the growth of infants, an anti-aging effect, a hematopoietic effect, a heart function improvement effect, a muscular fatigue inhibitory effect, an analgesic effect, the effect of reducing the side effects of anticancer agents, an immune enhancement effect, and an anti-stress effect.

However, velvet antler has side effects such as diarrhea. For this reason, in Chinese medicine, velvet antler is used in combination with herbs having an astringent effect, and in this case, the effects of velvet antler can be lost or reduced. In addition, in the process of finely cutting and decocting velvet antler in order to use it in herbs, the many active ingredients of velvet antler are lost.

Meanwhile, when velvet antler is fermented, a fermented velvet antler having an excellent immune enhancement effect can be obtained, and various fermentation methods and fermentation products are already known in the art.

Korean Patent Registration No. 808060 discloses a method of fermenting velvet antler using Bacillus lichemiformis (KCCM10885P) having the activity of fermenting velvet antler, and Korean Patent Registration No. 329365 discloses a velvet antler formulation and a preparation method thereof, in which velvet antler is fermented together with Galli Stomachichum Corium so that the absorption rate thereof is increased. In addition, Korean Patent Laid-Open Publication No. 10-2006-0128117 discloses a pharmaceutical composition for enhancing immunity, enhancing intestinal function and preventing and treating cancer, the composition containing, as an active ingredient, a fermented velvet antler extract obtained by sterilizing ground velvet antler in an about 10 to 60-fold weight of distilled water to make a velvet antler medium and culturing a bacillus P-92 strain in the velvet antler medium for 5-7 days, followed by sterilization and filtration.

However, the prior art methods that comprise fermenting ground or decocted velvet antler have shortcomings in that the fermentation process is complex, it is difficult to use velvet antler without any loss, and additional processes is required after fermentation because the fermentation products are mostly in a liquid state.

### DISCLOSURE

### Technical Problem

The present inventors have made various attempts to develop a preparation method, which has a high recovery rate and can maintain the shape of velvet antler even after fermentation. As a result, the present inventors have found that, when velvet antler is fermented using one or more mushroom concentrates selected from the group consisting of Polyozellus multiplex, Sarcodon asparacus, and Inonotus obliquus concentrates, the following shortcomings associated with the conventional fermentation processes can be solved.

In the conventional fermentation methods, velvet antler is ground or decocted and a velvet antler extract is used, and thus a significant portion of velvet antler is discarded, but the present invention is economic in that velvet antler can be used without any loss and ossified deer horns or bones can be used. In addition, in the conventional fermentation methods, the fermentation products are produced in a liquid state, and thus additional processes are required and it is difficult to process the fermentation products into various shapes, but in the present invention, velvet antler maintains its shape even after fermentation, and thus it can be changed into a desired shape for use or can be conveniently used in various shapes.

Meanwhile, in accordance with the present invention, velvet antler can be used without any loss, and thus all the active ingredients of velvet antler can be effectively taken. In addition, no offensive odor occurs in the fermentation process, and thus it is easy to handle and store velvet antler.

### Technical Solution

To achieve the above objects, the present invention provides a method for fermenting velvet antler, the method comprising wetting sliced velvet antler with one or more mushroom concentrates selected from the group consisting of Polyozellus multiplex, Sarcodon asparatus, and Inonotus obliquus concentrates and fermenting the wet velvet antler in grain.

The mushroom concentrates are prepared by boiling down mushroom powder in water, and the velvet antler wet with the mushroom concentrates is stacked on grain boiled with the mushroom concentrate and is fermented at 30-45° C. for 5-10 days.

### Advantageous Effect

The preparation method in accordance with the present invention enables velvet antler to be used without any loss and can prepare a fermentation product that contains all the active ingredients of velvet antler and maintains the shape of velvet antler. Meanwhile, in accordance with the preparation method of the present invention, ossified deer horns or deer bones can be used.

In addition, the fermented velvet anther prepared by the above method contains all the active ingredients of velvet antler and is easily deformed and added depending on the intended use. In addition, it has no offensive odor and is also tasty, and thus it is easy to take. Accordingly, the fermented velvet anther can be used in the form of capsules, pills, powder, extracts or the like and may be used in health functional foods, favorite foods such as milk products or confectionery, cosmetics and the like.

### BRIEF DESCRIPTION OF THE DRAWINGS

The above objects, other features and advantages of the present invention will become more apparent by describing the preferred embodiments thereof with reference to the accompanying drawings, in which:

FIG. 1 is a photograph of sliced velvet antler before fermentation.

FIG. 2 is a set of photographs showing a velvet antler fermented with a mushroom concentrate for 4 days in Example 1.

FIG. 3 is a set of photographs showing a velvet antler completely fermented with a mushroom concentrate in Example 1.

FIG. 4 is a set of photographs showing a velvet antler fermented with a Lentinus edodes concentrate for 4 days in Comparative Example 1.

FIG. 5 is a set of photographs showing a velvet antler fermented with a Lentinus edodes concentrate for 7 days in Comparative Example 1.

### PREFERRED EMBODIMENTS OF THE INVENTION

Now, preferred embodiments of the present invention will be described in detail with reference to the accompanying drawings. The matters defined in the description, such as the detailed construction and elements, are nothing but specific details provided to assist those of ordinary skill in the art in a comprehensive understanding of the invention, and the present invention is not limited to the embodiments disclosed hereinafter.

The present invention is directed to a method of fermenting velvet antler (or venison or deer bone) together with a mushroom concentration in order to increase the efficacy thereof and to a fermentation product obtained thereby. In accordance with the present invention, velvet antler is fermented in a state in which it was not concentrated or ground, and the shape thereof is maintained after fermentation. Thus, the loss of velvet antler is insignificant, and the fermented velvet antler has high efficacy and can be used in various shapes in subsequent processes.

In the present invention, velvet antler is wet with one or more mushroom concentrates selected from the group consisting of Polyozellus multiplex, Sarcodon asparatus and Inonotus obliquus concentrates, and is then fermented in grain.

Examples of the mushroom that may be used in the present invention include Lentinus edodes, Pleurotus ostreatus, Hericium erinaceum, Russula subdepallens, Gyrophora seculenta, Auricularia auricular, Phellius linteus, Ganoderma lucidum, Coriolus versicolor, Polyozellus multiplex, Inonotus obliquus, Sarcodon asparatus and the like. Among them, Polyozellus multiplex, Inonotus obliquus and Sarcodon asparatus have the highest fermentation efficiency. Particularly, a mushroom concentrate prepared by heating a mixture of powders of Polyozellus multiplex, Inonotus obliquus and Sarcodon asparatus in distilled water has the highest fermentation efficiency.

The mushroom concentrate is prepared by adding one or more mushroom powders selected from the group consisting of Polyozellus multiplex, Sarcodon asparatus and Inonotus obliquus powders to water and boiling down the aqueous solution until the volume thereof reaches the half. The amounts of water and Polyozellus multiplex powder are not specifically limited, but for example, 5-10 g of Polyozellus multiplex powder is preferably added to 400 mL of water. The aqueous solution of Polyozellus multiplex powder is preferably concentrated until the total volume thereof reaches 200 mL.

Sliced velvet antler is placed in and completely wet with the mushroom concentrate and stacked between boiled grains, after which it is fermented at 30-45° C. for 5-10 days. The time during which velvet antler is immersed in the concentrate is such that the moisture of the concentrate is absorbed. Specifically, the time is 5 minutes or shorter. When the grain is boiled with the mushroom concentrate, the fermentation effect is better. When the fermentation temperature is relatively high, the fermentation of velvet anther can be achieved even within 5 days, and when the fermentation temperature is relatively low, the fermentation time can be 10 days.

After fermentation, the velvet antler is naturally dried in an airy cool shade for about 1-2 days.

The kind of boiled grain is not limited, but grain whose humidity is easily controlled in accordance with a season is used in the present invention. All grains, including beans, barley, wheat, rice, buckwheat, rice bran, millet, Indian millet, oat, malt, chaff, mung beans and other grains, may be used in the present invention, but millet or wheat is preferably used in view of fermentation efficiency. When grains other than millet or wheat are used, fermentation efficiency is reduced. Particularly, in the case of the summer season, it is most preferable that raw buckwheat powder be added to millet or wheat to control the humidity. Buckwheat powder is added in an amount of 10-20 wt % based on the total weight of the grains. If buckwheat powder is added in an amount of less than 10 wt %, it will hardly show the effect of controlling the humidity, and if it is added in an amount of more than 20 wt %, fermentation will not easily occur.

For fermentation, straw is spread at the bottom, a basket having holes is placed thereon, and a layer of grain such as millet or wheat is formed in the basket to a height of 1-2 cm, after which the velvet anther wet with the mushroom concentrate is placed on the grain layer. Then, the grain layer is placed again on the velvet anther to the same height. A plurality of the grain layers with the velvet antler therebetween can be stacked, and preferably, three grain layers may be stacked. When a plurality of the grain layers are stacked, production efficiency will be increased, but fermentation efficiency is the highest when a single grain layer is formed. After the grain layer and the velvet antler layer have been alternately stacked as described above, the layers are preferably covered with a basket made of Korean paper or cotton such as gauze and are covered with straw.

Before the start of fermentation, the humidity of velvet antler is about 90%, because it has been wet with the mushroom concentrate. As fermentation progresses, the humidity decreases to 50-80%, and after the completion of fermentation, the humidity is decreased to about 20% by a drying step. After the completion of fermentation, the weight of the velvet antler also decreases to about the half of the weight before the start of fermentation.

Hereinafter, the present invention will be described in further detail with reference to examples. However, these examples are for illustrative purposes only and are not intended to limit the scope of the present invention.

### Example 1

Polyozellus multiplex, Sarcodon asparatus and Inonotus obliquus are mixed with each other at a ratio of 1:1:1, and 5 g of the mixed powder is added to 400 mL of water, heated to a temperature of 100° C. or higher and concentrated, thereby preparing 200 mL of a mushroom concentrate.

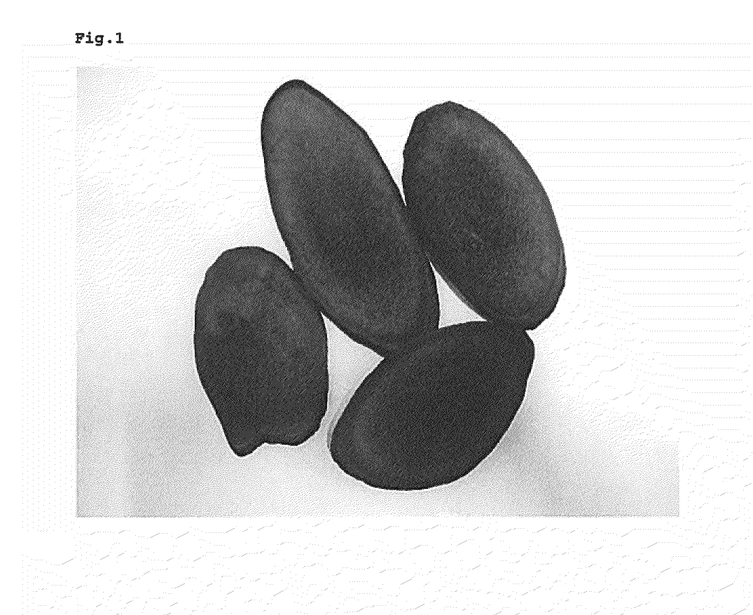

100 g of sliced velvet antler (see FIG. 1) was completely wet with the mushroom concentrate.

800 g of millet was boiled with the mushroom concentrate.

The boiled grain was spread to a thickness of 1.5 cm, the velvet antler wet with the mushroom concentrate was placed thereon, and the boiled grain was covered again on the velvet antler to a thickness of 1.5 cm, followed by fermentation at a fermentation room at 35-40° C. for 7 days.

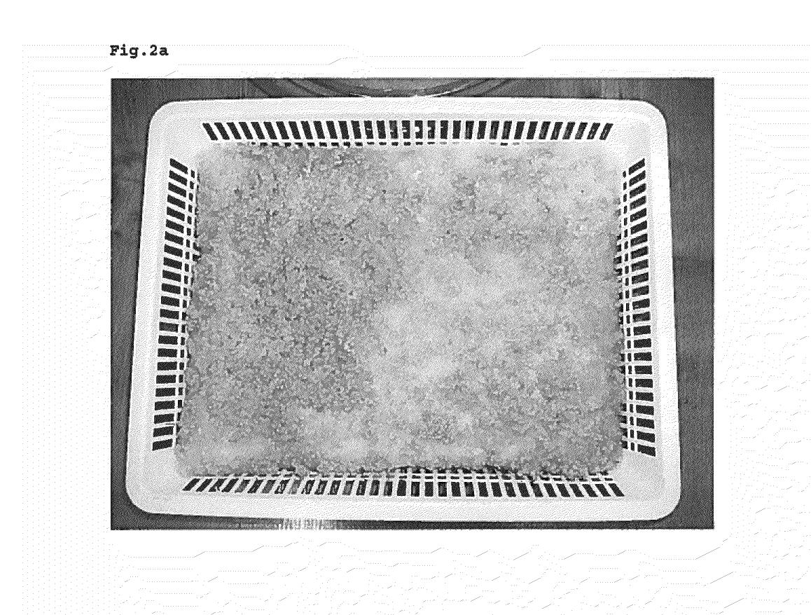

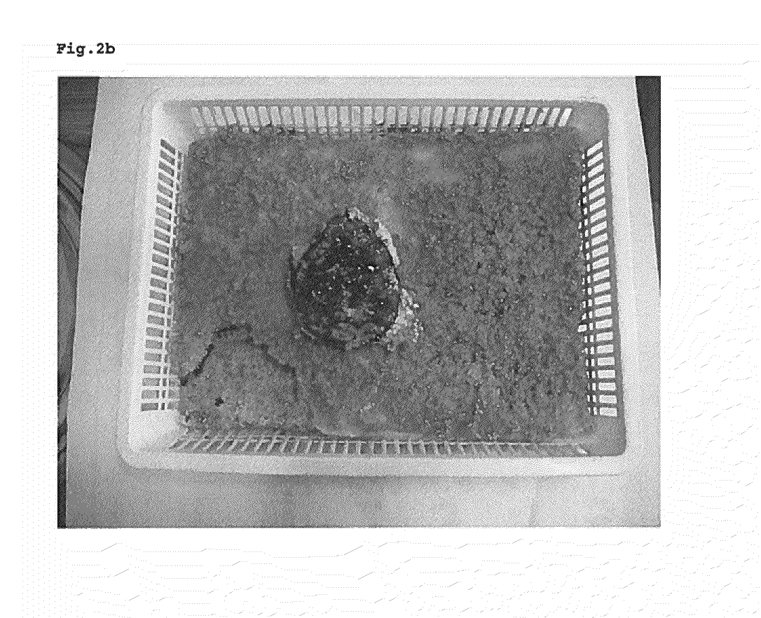

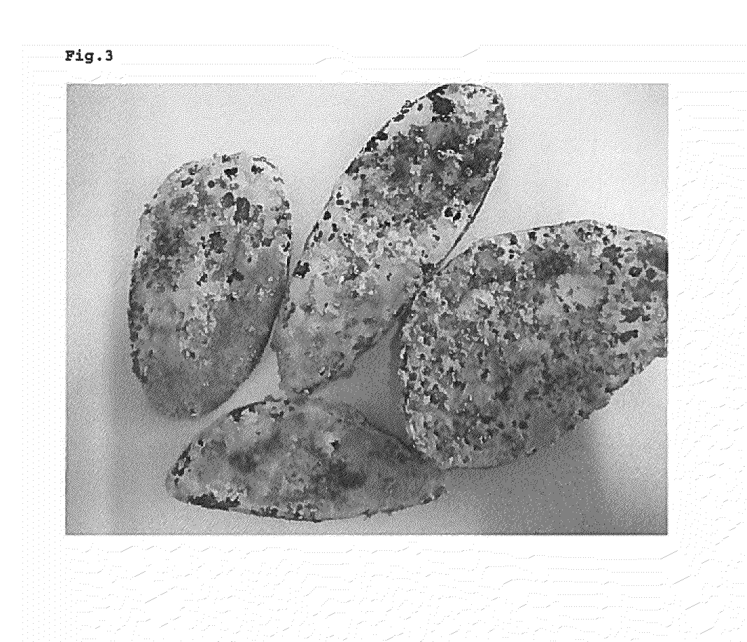

FIGS. 2 and 3 show photographs of the velvet antler obtained as described above. FIG. 2 shows photographs of the velvet antler at 4 days after the start of fermentation, FIG. 3 is a photograph of the velvet antler completely fermented at 7 days after the start of fermentation. In addition, FIG. 2a is a photograph of the velvet antler covered with millet, and FIG. 2b is a photograph of the velvet antler from which millet was removed.

### Example 2

5 g of Polyozellus multiplex powder was added to 400 mL of water and boiled down at a temperature of 100° C. or higher to prepare 200 mL of a Polyozellus multiplex concentrate.

100 g of sliced velvet antler was completely wet with the Polyozellus multiplex concentrate.

800 g of wheat was boiled with the Polyozellus multiplex concentrate.

The boiled grain was spread to a thickness of 1.5 cm, the velvet antler wet with the Polyozellus multiplex concentrate was placed thereon, and the boiled grain was covered again on the velvet antler to a thickness of 1.5 cm, followed by fermentation at a fermentation room at 35-40° C. for 7 days.

The velvet antler obtained as described above showed a completely fermented shape, like that in Example 1.

### Example 3

5 g of Inonotus obliquus powder was added to 400 mL of water and boiled down at a temperature of 100° C. or higher to prepare 200 mL of an Inonotus obliquus concentrate.

100 g of sliced velvet antler was completely wet with the Inonotus obliquus concentrate.

A mixture of 700 g of wheat and 100 g of buckwheat was boiled with the Inonotus obliquus concentrate.

The boiled grain was spread to a thickness of 1.5 cm, the velvet antler wet with the Inonotus obliquus concentrate was placed thereon, and the boiled grain was covered again on the velvet antler to a thickness of 1.5 cm, followed by fermentation at a fermentation room at 35-40° C. for 7 days.

The velvet antler obtained as described above showed a completely fermented shape, like that in Example 1.

### Example 4

5 g of Sarcodon asparatus powder was added to 400 mL of water and boiled down at a temperature of 100° C. or higher to prepare 200 mL of a Sarcodon asparatus concentrate.

100 g of sliced velvet antler was completely wet with the Sarcodon asparatus concentrate.

800 g of wheat was boiled with the Sarcodon asparatus concentrate.

The boiled grain was spread to a thickness of 1.5 cm, the velvet antler wet with the Sarcodon asparatus concentrate was placed thereon, and the boiled grain was covered again on the velvet antler to a thickness of 1.5 cm, followed by fermentation at a fermentation room at 35-40° C. for 7 days.

The velvet antler obtained as described above showed a completely fermented shape, like that in Example 1.

### Comparative Example 1

5 g of Lentinus edodes powder was added to 400 mL of water and boiled down at a temperature of 100° C. or higher to prepare 200 mL of a mushroom concentrate.

100 g of sliced velvet antler was completely wet with the mushroom concentrate.

800 g of millet was boiled with the mushroom concentrate.

The boiled grain was spread to a thickness of 1.5 cm, the velvet antler wet with the mushroom concentrate was placed thereon, and the boiled grain was covered again on the velvet antler to a thickness of 1.5 cm, followed by fermentation at a fermentation room at 35-40° C. for 7 days.

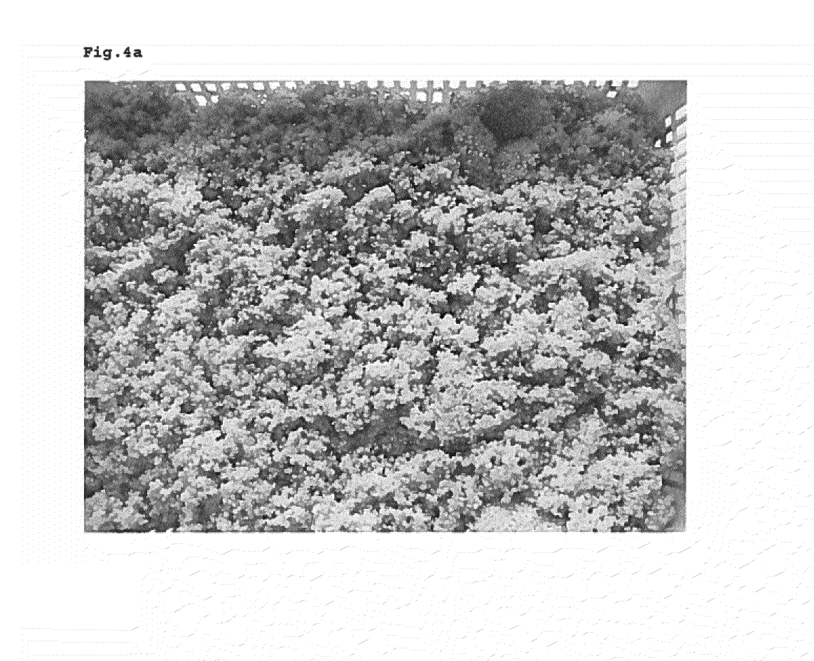

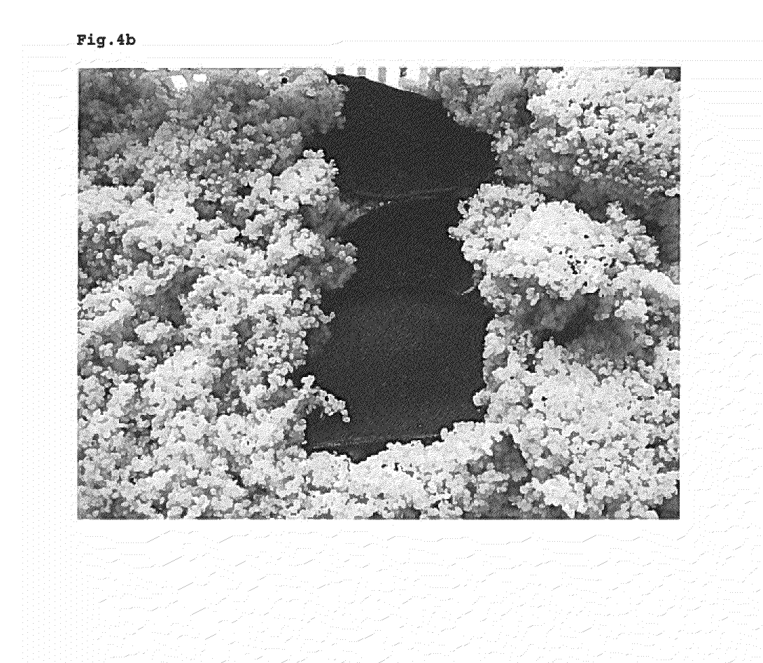

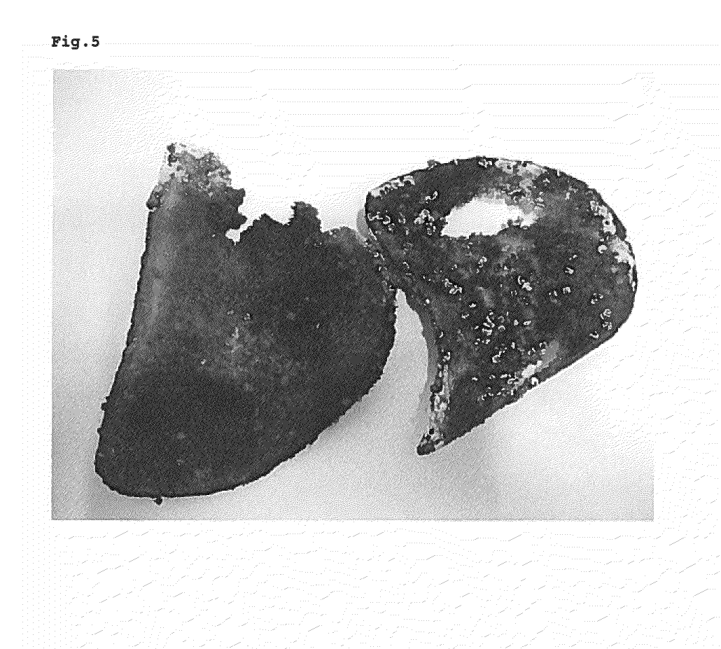

FIG. 4 shows photographs of the velvet antler at 4 days after the start of fermentation. FIG. 4a is a photograph of the velvet antler covered with the grain, and FIG. 4b is a photograph of the velvet antler from which the grain was partially removed. FIG. 5 shows the state of the velvet antler at 7 days after the start of fermentation. As can be seen therein, fermentation did not substantially occur.

### Comparative Example 2

5 g of Pleurotus ostreatus powder was added to 400 mL of water and boiled down at a temperature of 100° C. or higher to prepare 200 mL of a mushroom concentrate.

100 g of sliced velvet antler was completely wet with the mushroom concentrate.

800 g of wheat was boiled with the mushroom concentrate.

The boiled grain was spread to a thickness of 1.5 cm, the velvet antler wet with the mushroom concentrate was placed thereon, and the boiled grain was covered again on the velvet antler to a thickness of 1.5 cm, followed by fermentation at a fermentation room at 35-40° C. for 7 days.

The state of the velvet antler was observed at 7 days after the start of fermentation, and as a result, it could be seen that fermentation did not substantially occur, and the velvet antler had an offensive odor. The fermentation rate was less than 50%.

### Comparative Example 3

5 g of Tricholoma matsutake powder was added to 400 mL of water and boiled down at a temperature of 100° C. or higher to prepare 200 mL of a mushroom concentrate.

100 g of sliced velvet antler was completely wet with the mushroom concentrate.

800 g of wheat was boiled with the mushroom concentrate.

The boiled grain was spread to a thickness of 1.5 cm, the velvet antler wet with the mushroom concentrate was placed thereon, and the boiled grain was covered again on the velvet antler to a thickness of 1.5 cm, followed by fermentation at a fermentation room at 35-40° C. for 7 days.

The state of the velvet antler was observed at 7 days after the start of fermentation, and as a result, it could be seen that fermentation did not substantially occur, and the velvet antler had an offensive odor. The fermentation rate was less than 50%.

### Comparative Example 4

5 g of Polyozellus multiplex powder was added to 400 mL of water and boiled down at a temperature of 100° C. or higher to prepare 200 mL of a mushroom concentrate.

100 g of sliced velvet antler was completely wet with the mushroom concentrate.

800 g of rice was boiled with the mushroom concentrate.

The boiled grain was spread to a thickness of 1.5 cm, the velvet antler wet with the mushroom concentrate was placed thereon, and the boiled grain was covered again on the velvet antler to a thickness of 1.5 cm, followed by fermentation at a fermentation room at 35-40° C. for 7 days.

The state of the velvet antler was observed at 7 days after the start of fermentation, and as a result, it could be seen that the velvet antler did slightly smell, and the fermentation rate was less than 50%.

In Comparative Examples 1 to 3, fermentation was performed using different kinds of mushroom under the same conditions as those of the Example, and as a result, the velvet antler had an offensive odor and was not sufficiently fermented. In addition, it could be seen that the kind of grain was important in fermentation (see Comparative Example 4).

### INDUSTRIAL APPLICABILITY

The present invention provides fermented velvet antler that may be applied to healthy food, milk products, confectionery, cosmetics and the like in the form of capsules, pills, powders, extracts and the like.

While the present invention has been described in connection with the specific embodiments illustrated in the drawings, they are merely illustrative, and the invention is not limited to these embodiments. It is to be understood that various equivalent modifications and variations of the embodiments can be made by a person having an ordinary skill in the art without departing from the spirit and scope of the present invention. Therefore, the true technical scope of the present invention should not be defined by the above-mentioned embodiments but should be defined by the appended claims and equivalents thereof.

What is claimed is:

1. A method for fermenting velvet antler, the method comprising wetting velvet antler with one or more mushroom concentrates selected from the group consisting of Inonotus obliquus, Polyozellus multiplex, and Sarcodon asparatus, concentrates and fermenting the wet velvet antler.

2. The method of claim 1, wherein the velvet antler wet with the mushroom concentrate is fermented in boiled grain.

3. The method of claim 2, wherein the gain is either wheat or millet.

4. The method of claim 1, wherein the raw buckwheat powder is added to the grain in an amount of 10-20 wt % based on the total weight of the grain.

5. The method of claim 1, wherein the mushroom concentrate is prepared by boiling down one or more mushroom powders selected from the group consisting of Inonotus obliquus, Polyozellus multiplex, and Sarcodon asparatus powders in water.

6. The method of claim 1, wherein the grain is boiled using the mushroom concentrate.

7. The method of claim 1, wherein the fermentation of the wet velvet antler is performed at 30-45° C. for 5-10 days.

8. A fermented velvet antler prepared by the method of claim 1.

9. The fermented velvet antler of claim 8, which is in the form of a solid.
